(19) United States
(12) Patent Application Publication
(10) Pub. No.: US 2010/0268111 A1
(43) Pub. Date: Oct. 21, 2010

(54) MONITORING PLATFORM FOR WOUND AND ULCER MONITORING AND DETECTION

(75) Inventors: Darrel D. Drinan, San Diego, CA (US); Carl F. Edman, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: PhiloMetron, Inc., San Diego, CA (US)

(21) Appl. No.: 12/828,110

(22) Filed: Jun. 30, 2010

Related U.S. Application Data
(63) Continuation of application No. 11/219,348, filed on Sep. 2, 2005, now abandoned.
(60) Provisional application No. 60/606,778, filed on Sep. 2, 2004.

(51) Int. Cl. A61B 5/053; A61F 13/15

(52) U.S. Cl. 600/547; 602/43

(57) ABSTRACT

Systems and techniques for monitoring hydration. In one implementation, a method includes measuring an electrical impedance of a region of a subject to generate an impedance measurement result, and wirelessly transmitting the data to a remote apparatus. The probe with which impedance is measured may in the form of a patch adhesively secured to the subject.

### CROSS-REFERENCE TO RELATED APPLICATIONS

This application is a continuation of U.S. application Ser. No. 11/219,348 filed Sep. 2, 2005 and entitled MONITORING PLATFORM FOR WOUND AND ULCER MONITORING AND DETECTION which claims priority to U.S. Provisional Application Ser. No. 60/606,778 filed Sep. 2, 2004 and entitled “NON-INVASIVE MONITORING PLATFORM FOR DEHYDRATION, BLOOD LOSS, WOUND MONITORING, AND ULCER DETECTION,” the entire content of which is hereby incorporated by reference in its entirety.

### BACKGROUND

Many species of organisms are largely water. The amount and/or disposition of water in an individual organism (i.e., the hydration of the organism) has been correlated with the health of the individual organism. For example, an excess or a scarcity of water can be indicative of acute and/or chronic disease states. Changes in body composition such as percent fat content and the like can also result in changes in body water content.

Because the electrical impedance of an organism will vary with changes in water content, impedance measuring devices have been devised that are intended to provide indications of total body water based on measured body impedance. Although such devices have been found useful in some applications, the potential of bioimpedance data to supplement medical diagnosis and treatment has not been fully realized.

### SUMMARY

In one embodiment, the invention comprises a method of monitoring a wound comprising measuring electrical impedance of tissue proximate to the wound at two or more times during wound healing.

In another embodiment, the invention comprises a wound dressing. The wound dressing comprises an absorbent material adapted to absorb wound exudate, and a plurality of electrodes situated to apply an electric current and/or voltage to tissue proximate to the wound.

In another embodiment, methods of reducing incidence of pressure wounds and/or cutaneous ulcers in a patient comprises detecting susceptibility to pressure wounds or cutaneous ulcers by measuring electrical impedance of a region of the body of the patient.

### BRIEF DESCRIPTION OF THE DRAWINGS

FIG. 1 shows a probe for monitoring the hydration of an organism.

FIG. 2 shows another impedance measurement probe for monitoring the hydration of an organism.

FIG. 3A and 3B illustrate bandage impedance measurement probes.

FIG. 4 illustrates a patch impedance measurement probe.

FIGS. 5 and 6 illustrate a strap impedance measurement probe.

FIG. 7 shows a system for monitoring the hydration of an organism or portion thereof.

FIGS. 8A-8D show various applications of strap impedance measurement probes on a human body.

FIG. 9 shows an example of a model equivalent circuit of tissue proximate to a wound.

### DETAILED DESCRIPTION

As described above, most applications of bioimpedance have been directed to monitoring the impedance of the entire body or at least large portions of it. In accordance with some aspects of the present invention, however, more localized measurements are used to beneficial effect. In some embodiments, as explained in detail below, actual or potential wound or injury sites are locally monitored to improve treatment for people that have or are susceptible to wounds of various types.

In a variety of contexts, wounds form on individuals due to disease or condition, or are created from accident and other forms of injury. These may be cuts, burns, surgical sites, bed sores and the like. Various skin ulcers such as venous stasis, ulcers, diabetic foot ulcers, pressure ulcers, burn site wounds, and donor site wounds can emerge from chronic diseases or various injuries. These cutaneous ulcers are accompanied by a degradation of the dermal layers, and often are subject to infection and resistant to healing. These wounds may be treated by cleaning, bandages, topical antibiotics, etc. Infection is a common problem, and such infections may further imperil an individual, cause more pain, delay the healing process and result in amputation or death. In many situations, monitoring the progress of wound healing is a necessary part of a successful treatment protocol.

To make such monitoring more effective and pain free, the wound or ulcer site is covered with a dressing that integrates one or more physical or biological parameter sensors. Advantageously, sensors for monitoring impedance of tissue proximate to the wound are provided to detect changes in local tissue edema. Skin temperature and/or heat flux from the skin may also be monitored by sensors on the dressing. Atypical changes in impedance, skin temperature, and or dermal heat flux may be detected and may be used to alert the patient and/or attending clinicians to possible healing interruption, infection or hemorrhaging at the wound site and facilitate early detection and treatment.

Similar apparatus used to monitor wound infections can also be used to monitor individuals at risk for wounds such as pressure sores and cutaneous ulcers. The apparatus may measure the typical changes in hydration and temperature of the tissue that is known to occur prior to the emergence of an ulcer or conditions that promote an ulcer. Early detection of the emergence of an ulcer would ensure that proper care is initiated quickly, thereby reducing the incidence of ulceration, infection and other complications.

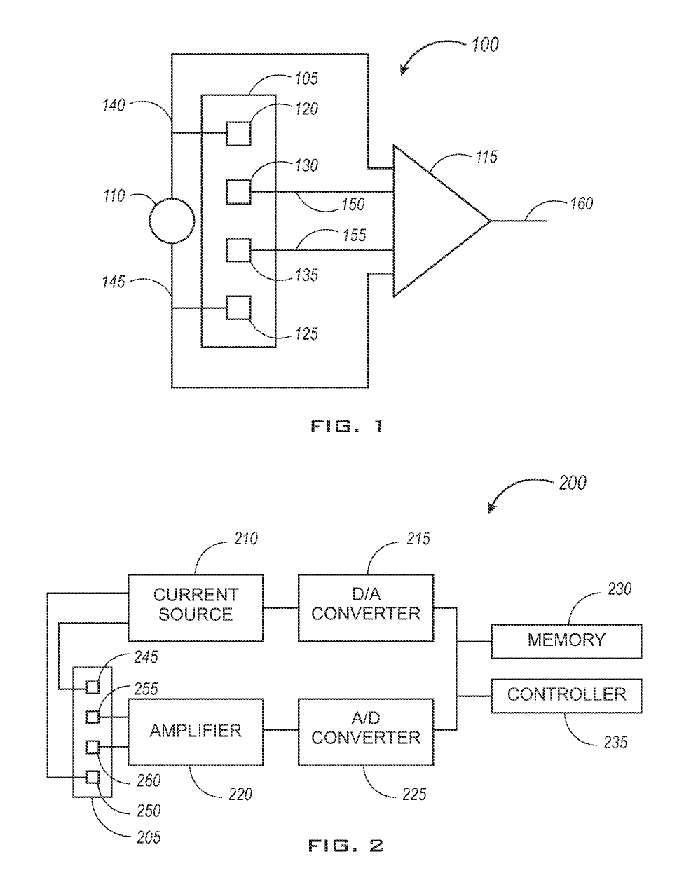

FIG. 1 shows a probe 100 for monitoring the hydration of an organism. Probe 100 includes a body 105, an energy source 110, and a sensing circuit 115. Body 105 can be a flexible member in that it can be contoured to follow the skin surface or other portion of an organism, such as, for example, a patch or strap. Body 105 supports probe/organism interfaces 120, 125, 130, 135 which apply or exchange energy with the subject and which sense energy exchange parameters in a way to measure the impedance of a region of the subject. In most embodiments, interfaces 120, 125 130, 135 will be electrodes adapted to exchange electrical energy with a human, although some optical element adapted to illuminate a human may also be possible. Typically, two of the interfaces 120, 125 are used to force current flow from one point on the subject to a second point on the subject. The other two interfaces 130, 135 are used to measure the voltage across two points on the subject. In most instances, voltage measurements will employ structures in direct contact with the skin surface. In certain applications, such voltage measurements may employ one or more contactless, voltage sensitive electrodes, e.g. capacitively coupled electrodes. It may be noted that the current application points and the voltage measurement points in these embodiments can be the same, adjacent to one another, or at significantly different locations.

Energy source 110 can be, e.g., an optical energy source or an electric energy source. For example, energy source can be one or more alternating and/or direct current and/or voltage source. Energy source 110 is connected to inputs 120, 125 by leads 140, 145. Leads 140, 145 can conduct energy generated by source 110 for exchange with the portion of the organism coupled to main body 105. For example, leads 140, 145 can be electrical wires capable of carrying an electric current for exchange with the portion of the organism, or leads 140, 145 can be optical waveguides capable of carrying light for exchange with the portion of the organism followed by main body 105.

In one electrical embodiment, a sensing circuit 115 comprises a differential amplifier connected to electrodes 120, 125 by leads 140, 145 and to electrodes 130, 135 by leads 150, 155. Leads 140, 145 can conduct voltage across source 110 to amplifier 115. Leads 150, 155 can conduct voltage across electrodes 130, 135 as another input to the amplifier 115. Amplifier 115 can sense voltages across electrodes 130, 135 and electrodes 120, 125 to generate one or more results 160. It will be appreciated that amplifier 115 could be implemented as two or more amplifiers that separately sense relative voltages across any desired electrode pairs. Current sensing could also be implemented to directly measure the current output from source 110.

In operation, main body 105 flexes to follow a portion of an organism and maintain inputs 120, 125 and outputs 130, 135 so that they can exchange energy with the followed portion. Source 110 generates one or more types of energy that is conducted over leads 140, 145 through interfaces 120, 125 and exchanged with the followed portion of the organism. In turn, interfaces 130, 135 sense one or more energy exchange parameters from the followed portion. Sensing circuit 115 generates a result 160 based on the sensed signals. Result 160 reflects, at least in part, the hydration of the monitored organism.

Probe 100 can generate result(s) 160 continuously or intermittently over extended periods of time. For example, result 160 can be a subset of the comparisons of the sensed parameters at interfaces 130, 135 with the amount of energy input at inputs 120, 125, or result 160 can be all such comparisons. For example, result 160 can be intermittent samples of voltages from the results of continuous application of a substantially constant current. As another example, result 160 can be periodic (e.g., every 5 to 30 minutes, such as every 10 minutes) results of successive, shorter duration current applications.

FIG. 2 shows one circuit implementation of a bioelectric impedance probe 200 for monitoring the hydration of a portion of an organism. Bioelectric impedance spectroscopy is a measurement technique in which the electrical impedance of all or a portion of an organism is measured. When the conductivity of the entirety of an organism is measured such as by passing current from one ankle to an opposite wrist or between both hands, this can be referred to as whole body bioelectric impedance spectroscopy. When the electrical impedance of a portion of an organism is measured such as by a cluster of more locally placed electrodes, this can be referred to as segmental (or regional) bioelectric impedance spectroscopy. In either case, the measured electrical conductivity can reflect the hydration of the measured organism or the measured portion of the organism.

Bioelectric impedance spectroscopy generally involves the exchange of electrical energy with the organism. The exchanged electrical energy can include both alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz.

Different frequencies of electrical energy can be used to measure conductivity in different portions of the organism. For example, in some organisms, lower frequency electrical energy may be conducted preferentially through tissues having fewer membranous components whereas higher frequencies may be conducted through a larger variety of tissues. In many cases, it is advantageous to make impedance measurements at two or more different frequencies in the same region. As explained further below, DC measurements can help characterize impedance over the skin surface. Thus, measurements at different frequencies made by a single probe can provide information regarding both the amount and disposition of water within a probed organism or within a probed portion of the organism.

Referring again to FIG. 2, bioelectric impedance spectroscopy probe 200 includes a body 205, a current source 210, a digital-to-analog converter 215, an amplifier 220, an analog-to-digital converter 225, a memory 230, and a controller 235. Body 205 is a flexible member that supports two working electrodes 245, 250 and two sensing electrodes 255, 260. Body 205 can be flexible enough to follow a portion of the human body to maintain electrodes 245, 250, 255, 260 in contact with that portion. The followed portion can include skin surfaces, mucosal surfaces in the mouth and/or nasal passages, and other body passages or orifices. Body 205 can be sized to probe the conductivity of the entirety of an organism and thus perform whole body bioelectric impedance spectroscopy. In some advantageous embodiments described in detail herein, body 205 is sized to probe the conductivity of a portion of an organism and thus perform segmental bioelectric impedance spectroscopy.

Working electrodes 245, 250 can be adapted to conduct current through or along the probed portion of the monitored organism. Sensing electrodes 255, 260 can be adapted to measure the potential of locations in the probed portion of the monitored organism. Electrodes 245, 250, 255, 260 are generally electrically conductive in that their electrical impedance is relatively small when compared to the electrical impedance of the monitored portion of an organism at the probed frequency. For example, electrodes 245, 250, 255, 260 can include metals, sintered metallic composites, conductive polymers, gels, carbon-based materials, silicon materials, electrically conductive microneedles, conductive solutions, or combinations thereof. In one implementation, electrodes 245, 250, 255, 260 are electrically conductive adhesive gel electrodes such as the RED DOT electrodes available from 3M Corp. (St. Paul, Minn.).

Electrodes 245, 250, 255, 260 can be supported by body 205 on the outer surface of the skin of a monitored organism. Alternatively, electrodes 245, 250, 255, 260 can be supported by body 205 beneath the skin of a monitored organism. For example, electrodes 245, 250, 255, 260 can be supported subdermally or electrodes 245, 250, 255, 260 can be supported on transdermal elements such as microneedles that penetrate the skin. When placed on the skin surface, electrodes 245, 250, 255, 260 can advantageously be each supported by body 205 at positions that are separated from one another by more than approximately ten times the thickness of the skin. When hydration is monitored in humans, electrodes 245, 250, 255, 260 that are above the skin can each generally be supported at positions that are separated from one another by more than 2.5 millimeters. In one implementation, the distance between working electrodes 245, 250 is greater than 1 cm. For embodiments that include a localized cluster of electrodes on one or more patches secured to the skin, the distance between electrodes is advantageously less than about 25 cm so that the impedance measurement is focused regionally on the subject. Such regional measurements have been found to produce useful data that can be generated and distributed with convenient apparatus.

In one implementation, working electrodes 245, 250 are different than sensing electrodes 255, 260. For example, working electrodes 245, 250 can be larger than sensing electrodes 255, 260 and/or made from different materials. In other implementations, sensing electrodes 255, 260 may be contactless electrodes, e.g. capacitively coupled electrodes (Quasar, San Diego, Calif.) while working electrodes 245, 250 are contact-based electrodes, e.g. RED DOT electrodes.

Current source 210 is a source of alternating and/or direct electrical current. As deployed in probe 200, current source 210 can drive electrical current from working electrode 245 to working electrode 250 through and/or along a monitored organism. In one implementation, current source 210 is capable of driving between 10 microamperes and 10 milliamperes, preferably between 100 microamperes and 1 milliamperes, of one or more frequencies of alternating and/or direct current through or along electrical impedances characteristic of humans. Typically, current is held at a known or measured substantially constant value, and voltage is measured to provide an impedance value. It is also possible to apply a constant voltage and measure the amount of current. Digital-to-analog converter 215 can be an integrated circuit or other electronic device that converts a digital signal into a corresponding analog signal. As deployed in probe 200, digital-to-analog converter 215 can convert digital control signals from controller 235 into analog control signals to control the output of electrical current from current source 210.

Amplifier 220 can be a differential voltage amplifier in that it amplifies a voltage difference on sensing electrodes 255, 260. This voltage difference results from current source 210 driving electrical current from working electrode 245 to working electrode 250 through and/or along the monitored organism. Analog-to-digital converter 225 can be an integrated circuit or other electronic device that converts this sensed voltage difference into a corresponding digital signal for reading by controller 235 and/or storage in memory 230.

Memory 230 can be a data storage device that can retain information in machine-readable format. Memory 230 can be volatile and/or nonvolatile memory. For example, memory 230 can be a RAM device, a ROM device, and/or a memory disk.

Controller 235 is a device that manages the generation and flow of data in probe 200. Controller 235 can be hardware configured to perform select operations or a data processing device that performs operations in accordance with the logic of a set of machine-readable instructions. In some implementations, controller can receive information related to the management of the generation and flow of data in probe 200 via one or more input devices. In some implementations, controller 235 can output information from probe 200 via one or more output devices. Custom ASICs or gate arrays can be used, as well as commercially available microcontrollers from, for example, Texas Instruments and Motorola.

The operations performed by controller 235 can include regulating the timing of hydration measurements and the timing of the transmission of hydration measurement results, logic operations, signal processing, and data analysis. For example, data analysis can be used to determine the bioelectric impedance of portions of a monitored organism. For example, equivalent circuit impedance analysis in the time or frequency domain can be performed. Instructions for performing such operations can be stored in a read only memory portion of memory 230, temporary values generated during such operations can be stored in a random access portion of memory 230, and the results of operations can be stored in a non-volatile portion of memory 230.

In operation, current source 210 drives one or more frequencies of alternating and/or direct current between working electrodes 245, 250 and through the subject organism. Amplifier 220 buffers and amplifies the potential difference between sensing electrodes 255, 260. Analog-to-digital converter 225 converts this signal into a digital form that can be received by controller 235 for storage at memory 230, as appropriate. In some implementations, controller 235 may control source 210 to change the frequency and/or magnitude of current generated. The control of source 210 can be performed in light of the magnitude of the signal(s) output by amplifier 220 and/or in light of instructions received by controller 235 over one or more input devices.

In accordance with some aspects of the present invention, the conductivity of a region of the organism that has a wound or is susceptible to wound formation (e.g. a diabetic cutaneous ulcer) is monitored by the probe. Generally speaking, wounds that are healing normally become drier, and the impedance and reactance of the region increases. Infected, open, interrupted healing, or draining wounds tend to have lower regional electric impedances. This can be monitored without bandage removal and visual inspection using a bandage incorporating impedance measurement sensing electrodes and/or circuits.

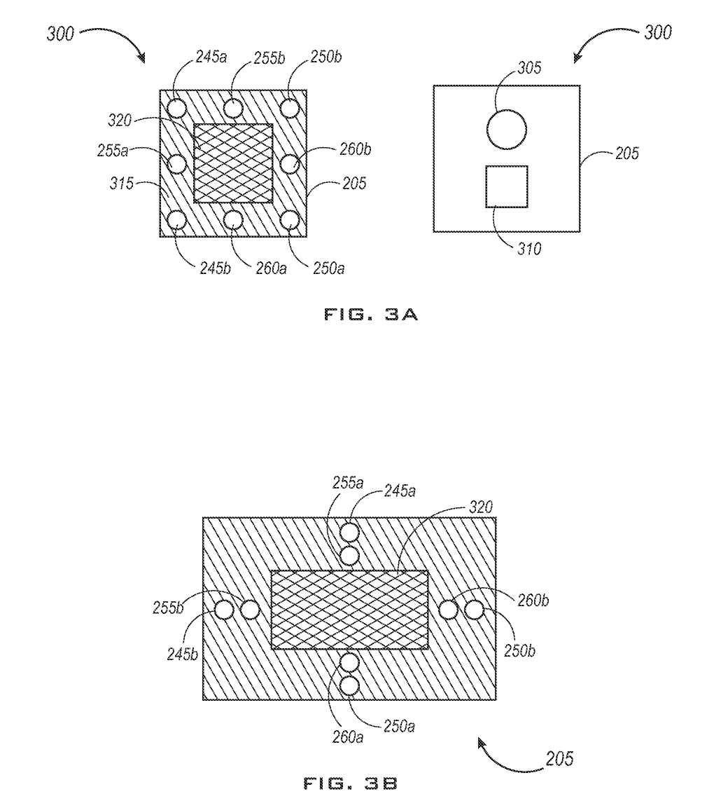

FIG. 3 shows one implementation of an impedance measuring probe suitable for use in wound monitoring. On the left is a view of the bottom side of the bandage probe, and on the right is a top view. Electrodes 245, 250, 255, 260 are provided around the periphery of the bandage. In one embodiment, when current is provided with electrode pairs 245a and 250a, voltage is measured across pair 255a and 260a and across pair 255b and 260b. If current is provided with electrode pairs 245b and 250b, voltage is measured across pair 255a and 255b and across pair 260a and 260b. It will be appreciated that various pairs of electrodes can be used for current supply and voltage measurements. During operation, the impedance of the tissue proximate to the wound is measured. In this context, “tissue proximate to the wound” includes actual wounded tissue, as well as tissue below and around the wound. Depending on the placement of the electrodes and the applied signal, the impedance of various current paths of different directions, depths, etc. can be explored and possibly mapped or characterized.

Additional sensors such as thermocouples or thermisters and/or heat flux sensors can also be provided, but are not shown in FIGS. 3A and 3B, to provide measured values useful in analysis. In general, skin surface temperature will change with changes in blood flow in the vicinity of the skin surface of an organism. Such changes in blood flow can occur for a number of reasons, including thermal regulation, conservation of blood volume, and hormonal changes. In one implementation, skin surface measurements of temperature or heat flux are made in conjunction with hydration monitoring so that such changes in blood flow can be detected and appropriately compensated for.

Probe 300 can be self-powered in that main body 205 can include (in addition to electrodes 245, 250, 255, 260) a portable power source, such as a battery 305. Advantageously, although not necessarily, circuitry on the probe 300 is adapted to perform at least some of the signal generation and processing, control, and data storage functions of current source 210, a digital-to-analog converter 215, an amplifier 220, an analog-to-digital converter 225, a memory 230, and a controller 235 without input from a fixed device. For example, the bandage probe 300 can be borne by the monitored organism over the wound being monitored. Circuitry 310 can be, e.g., an application specific integrated circuit (ASIC) adapted to perform these functions. Circuitry 310 can also be a data processing device and/or one or more input/output devices, such as a data communication device.

Main body 205 also advantageously includes an adhesive 315. Adhesive 315 can be adapted to adhere to the skin surface of the monitored organism and thereby maintain electrodes 245, 250, 255, 260 in contact with the portion of an organism followed by main body 205.

The bandage 300 allows a monitored organism to be ambulatory while hydration monitoring occurs. This allows for data collection to be extended beyond periods of confinement. Thus, hydration monitoring can be continued while an organism participates in various activities at different locations.

Placement of the bandage probe 300 at appropriate locations on the body would facilitate monitoring of disease states such as cutaneous ulcers in the locations where the bandage probe has been placed. Bandage probe 300 can be incorporated into a wound dressing anywhere on the body. In these cases, the probe is advantageously combined with an absorbent material 320 such as gauze or the like to absorb wound exudates. The gauze may be incorporate one or more pharmaceutical compounds or agents in the gauze, in a delivery device, or in a resorbable delivery matrix for example. An oxygen supply device may also be provided to supply oxygen to the wound. Any of these may be provided to enhance healing and/or inhibit infection. In addition, by monitoring of the wound without removal of the bandage, incidental exposure to bacteria, etc. in the wound vicinity may be minimized. In some embodiments, compound delivery from a device on the bandage 300 or located elsewhere on the organism may be initiated by circuitry 310 in response to measured values.

FIG. 3B shows a rectangular embodiment with different electrode placement. In this embodiment, electrodes 245 and 250 can be used to supply currents, and electrodes 250 and 260 can be used to measure voltages.

Multiple sets of sensing electrodes can be used to measure hydration of the region. Potential differences generated between different electrode pairs during current flow in different directions can be used to gain information about the conduction of current in the entire region. A measurement of multiple potential differences between more than two sensing electrodes can also be used, e.g., to make cross measurements and ratiometric comparisons that can be used to monitor hydration while aiding in calibration and helping to account for measurement variability such as temperature changes, changes in the position of the monitored individual, and movement of the device over time.

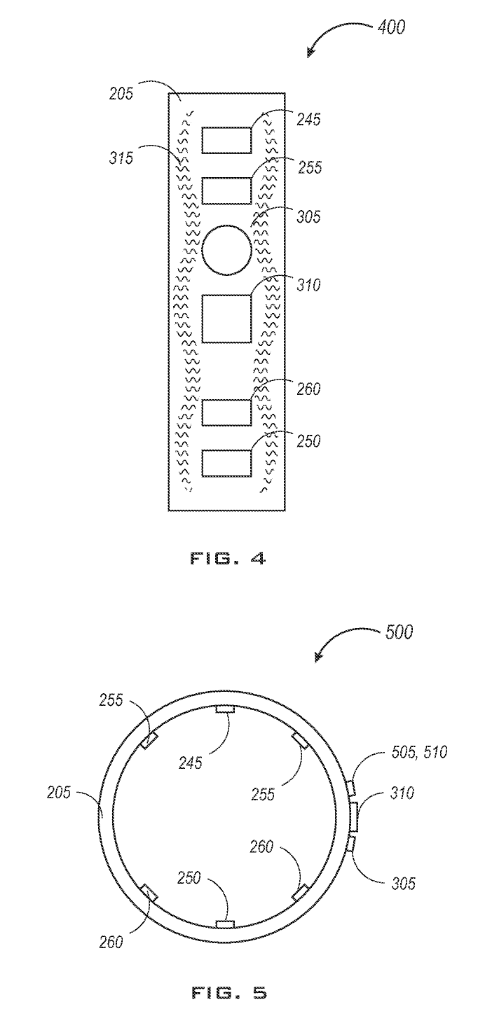

FIGS. 4, 5, and 6 illustrate impedance measurement probes that can be useful to detect a subject's susceptibility to wounds such as pressure sores and diabetic foot ulcers. Susceptibility to and onset of such wounds can be detected by detecting changes in hydration state of the region prior to the onset of the sore or ulcer or that otherwise compromise the tissue structure leading to increased susceptibility to injury thereby resulting in ulcer or other wound formation. FIG. 4 illustrates an impedance measurement probe in the form of a patch 400. FIGS. 5 and 6 show another implementation of an impedance measurement strap probe 500. Main body 205 of strap probe 500 is a strap or a belt that can form a loop to encircle the body, or a portion of the body, of a monitored individual. Such an encirclement can maintain electrodes 245, 250, 255, 260 in contact with the encircled portion. In addition to working electrodes 245, 250, two sets of sensing electrodes 255, 260, battery 305, and circuitry 310, main body 205 also includes a data communication device 505 having a transceiver 510. Data communication device 505 can be a wireless communication device that can exchange information between circuitry 310 and an external entity.

A wireless data link can carry information using any of a number of different signal types including electromagnetic radiation, electrical signals, or acoustic signals. For example, data communication device 505 can be a radio frequency communication device. Transceiver 510 can be an assembly of components for the wireless transmission and reception of information. The components can include, e.g., an RF antenna. The wireless receiver/transmitter circuitry can be made part of any embodiment described herein.

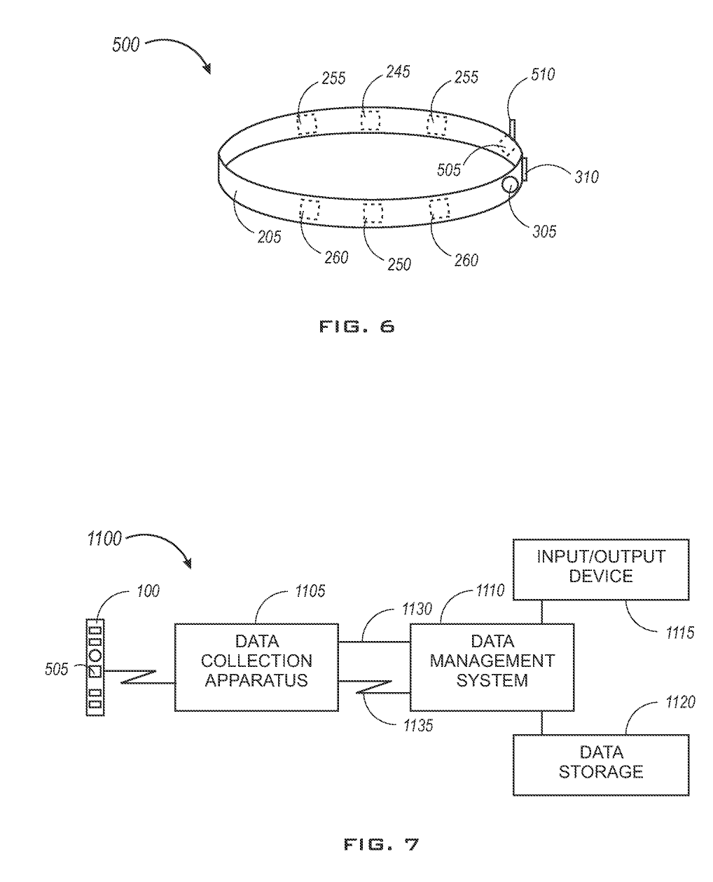

FIG. 7 shows a system 1100 for monitoring the hydration of an organism. System 1100 includes one or more probes 100 along with one or more data collection apparatus 1105, a data management system 1110, an input/output device 1115, and a data storage device 1120. Probe 100 includes a wireless data communication device 505 that is capable of establishing a wireless data link 1125 with data collection apparatus 1105. Wireless data link 1125 can transmit data using any of a number of different signals including electromagnetic radiation, electrical signals, and/or acoustic signals. When probe 100 is subdermal, data link 1125 can be a transdermal link in that data link 1125 conducts data along a path through the skin.

The data communicated along wireless data link 1125 can include a probe identifier. A probe identifier is information that identifies probe 100. Probe 100 can be identified, e.g., by make or model. Probe 100 can also be identified by a unique identifier that is associated with a single individual probe 100. The probe identifier can include a serial number or code that is subsequently associated with data collected by probe 100 to identify that this data was collected by probe 100. In some embodiments, each individual electrode, or a patch or strap containing a set of electrodes incorporates an integrated circuit memory having a stored unique or quasi-unique electrode/patch identifier. An interface between the patch or electrodes and the communication device 505 can be implemented so that the communication device 505 can send electrode or patch identifiers as well as a separate identifier for the other electronics coupled to the patch. In this way, different parts of the probe can be separately replaced, while still allowing complete tracking of the physical data generation, analysis, and communication apparatus used to gather all impedance data.

The data communicated along wireless data link 1125 can also include messages to probe 100. Example messages, include commands to change measurement and/or data analysis parameters and queries regarding the status and/or operational capabilities of the probe. Data communication along wireless data link 1125 can also include information related to the initialization and activation of probe 100. Initialization can include the communication of a probe identifier to data collection apparatus 1105. Initialization can also include the commencement of measurement activities including, e.g. the start of an internal clock that regulates the timing of hydration measurements and the transmission of hydration measurement results. Such data communication can be conducted as an ongoing dialogue with data collection apparatus 1105.

Data collection apparatus 1105 is a device that generally supplements probe 100 by including components and/or features that complement the components and/or features of probe 100. For example, such components or features may be too large, too memory intensive, require too sophisticated data processing, and/or only be used too intermittently to be included on probe 100. Data collection apparatus 1105 can be a portable device in that data collection apparatus 1105 can be moved from a fixed location and perform at least some functions without input from a fixed device. For example, data collection apparatus 1105 can be a handheld device that can be borne by a monitored individual.

Returning to FIG. 7, system 1100 can include a wired data link 1130 and/or a wireless data link 1135 for the exchange of data between data collection apparatus 1105 and data management system 1110. Wired data link 1130 can terminate at a connector port 1274 on data collection apparatus 1105, and wireless data link 1135 can terminate at transceiver 1270 on data collection apparatus 1105.

Wireless data link 1125, wired data link 1130 and wireless data link 1135 can exchange data in accordance with one or more communication protocols. The communication protocols can determine the format of the transmitted information and the physical characteristics of the transmission. Communication protocols can also determine data transfer mechanisms such as synchronization mechanisms, handshake mechanisms, and repetition rates. The data structures of the protocol may impact the rate of data transfer using the protocol. Data can be organized in blocks or packets and transmissions can be made at specified intervals. For example, a transmission block can include synchronization bits, an address field that includes information identifying the data source, a data field containing the hydration monitoring data, and a checksum field for testing data integrity at the receiver. The length of a data block can vary, e.g., to reduce power consumption and increase device lifetime. The same data can be transmitted multiple times to ensure reception.

In one implementation, exchanged data is organized in packets that include four sections, namely, a header section, a 64 bit address section that includes a probe identifier identifying a probe 100 (and/or an electrode or electrode set identifier), an encrypted data section, and a check-sum or error correction section. The data section can be encrypted using an algorithm that relies upon the address section.

Probe 100, data collection apparatus 1105, and data management system 1110 can all confirm a successful exchange of data using a confirmation such as an electronic handshake. An unsuccessful exchange of data can be denoted by transmission of an error message, which can be responded to by a retransmission of the unsuccessfully exchanged data.

In some implementations, probe 100, data collection apparatus 1105, and data management system 1110 can exchange data at a number of different frequencies. For example, when system 1100 includes multiple data collection apparatus 1105, each data collection apparatus 1105 can transmit data over wireless data link 1135 using a different frequency carrier. As another example, when system 1100 includes multiple probes 100, each probe 100 can transmit data over wireless data link 1125 using a different frequency carrier. It will be appreciated that a variety of multiple access techniques such as time or code division, could be alternatively used.

The data communicated along wireless data link 1125, wired data link 1130, and wireless data link 1135 can be encrypted in whole or in part. The encryption can be symmetric or asymmetric. The encryption can rely upon encryption keys based on the probe identifier or on alphanumeric codes transmitted with the encrypted data. The encryption may be intended to be decrypted by a specific probe 100, a specific data collection apparatus 1105, or a specific data management system 1110. In one implementation, data communicated along wired data link 1130 is encrypted using 128 bit encryption at the SSL layer of the TCP/IP protocol.

Both proprietary and public protocols can be used to exchange data between probe 100, data collection apparatus 1105, and data management system 1110. For example, the global system for mobile communications (GSM), Bluetooth, and/or the internet protocol (IP) can be used.

In one implementation, wireless link 1125 is a spread-spectrum RF signal at wireless medical band frequencies such as the Medical Implant Communications Service (MICS) (400-406 MHz) or the Wireless Medical Telemetry Service (WMTS) (609-613 MHz and 1390-1395 MHz).

Data management system 1110 is a data processing device that conducts operations with the data collected by probe 100 that relates to hydration of the organism. The operations can be conducted in accordance with the logic of instructions stored in machine-readable format. The conducted operations can include the processing of such data, the display of such data, and the storage of such data.

Data management system 1110 can be remote from data collection apparatus 1105 in that data management system 1110 need not be part of a local data communication network that includes data collection apparatus 1105. For example, data management system 1110 can be a data processing apparatus that is accessible by one or more medical personnel.

The processing of data by data management system 1110 can include data analysis to identify disease states in monitored organisms or problems with the monitoring. For example, data management system 1110 can perform impedance analysis using model equivalent circuits to determine hydration levels at different locations in a monitored organism.

The display of data by data management system 1110 can include the rendition of the results of hydration monitoring on one or more input/output devices 1115. Input/output device 1115 can include visual, auditory, and/or tactile display elements that can communicate information to a human user (such as medical personnel). For example, input/output device 1115 can include a monitor, a speaker, and/or a Braille output device. Input/output device 1115 can also include visual, auditory, and/or tactile input elements such as a keyboard, a mouse, a microphone, and/or a camera. Input/output device 1115 can thus render visual, auditory, and/or tactile results to a human user and then receive visual, auditory, and/or tactile input from the user.

The storage of data by data management system 1110 can include the storage of the results of hydration monitoring on one or more data storage devices 1120 that retain information in machine-readable format. Data storage devices 1120 can include volatile and/or nonvolatile memory. For example, data storage devices 1120 can be a RAM device, a ROM device, and/or a memory disk.

In operation, all or some of the constituent components of system 1100 can operate in one or more operational stages. For example, during a test stage, the constituent components of system 1100 can test themselves to determine that they are functional. For example, probe 100 and data collection apparatus 1105 can confirm that they are capable of exchanging data along link 1125, and data collection apparatus 1105 and data management system 1110 can confirm that they are capable of exchanging data along one or more of links 1130, 1135. As another example, probe 100 can confirm that inputs 120, 125 and outputs 130, 135 are properly positioned relative to a monitored organism. For example, when inputs 120, 125 and outputs 130, 135 are electrodes 245, 250, 255, 260, probe 100 can confirm that electrodes 245, 250, 255, 260 are in electrical contact with the followed portion of the monitored organism.

During a setup stage, parameters relating to the monitoring of the hydration of an individual can be arranged. For example, a probe 100 can determine the baseline measurement result for a given hydration level in a portion of a monitored organism and adjust monitoring parameters accordingly. The standard response can be indicative of the absence of a disease state or of the absence of progression in a disease state. Changes in the baseline impedance measurements can result from changes in factors unrelated to a disease state. For example, changes in the baseline impedance measurements can result from different skin thicknesses, body compositions, or other differences between two locations. Measurements made at the different locations can be normalized to account for such differences in baseline measurements. Such a normalization can include adjustments in gain and/or adjustments in offset. Gain adjustments may be based on the absolute value of the impedance measurement(s), the impedance difference(s) observed at the old and the new locations, or combinations thereof. Offset adjustments can generally be made after gain adjustments and can be based on absolute impedance values and/or other factors. Alternatively, analysis thresholds used to identify disease states can be adjusted. For example, the input signal level can be increased to accommodate dry skin and high transdermal impedances. Data collection apparatus 1105 can receive user input over one or more of local user input portion 1205, wireless data communication portion 1215, and wired data communication portion 1217. The received input can identify monitoring parameters that are to be adjusted, such as the level at which an alert is to be sounded at probe 100 and/or data collection apparatus 1105. Data management system 1110 can also receive user input relating to the arrangement of monitoring parameters. For example, data management system 1110 can receive input from medical personnel over input/output device 1115 indicating that hydration measurement results are to be transmitted by probe 100 to data collection apparatus over link 1125 once every four hours. This timing parameter can be relayed from data management system 1110 over link 1130 to data collection apparatus 1105 which relays the timing parameter over wireless link 1125 to probe 100.

Parameters relating to the communication of information over one or more of links 1125, 1130, 1135 can also be arranged during a setup stage. For example, the constituent components of system 1100 can select communication protocols or parameters for communication protocols.

During a synchronization stage, clocks in two or more of probe 100, data collection apparatus 1105, and data management system 1110 are synchronized to enable synchronous data transmission along one or more of links 1125, 1130, 1135. For example, in one implementation, data collection apparatus 1105 transmits synchronization characters to data management system 1110 over wired data link 1130. Data management system 1110 can receive the synchronization characters and compares the received characters with a synchronization pattern. When the received characters correspond sufficiently with the synchronization pattern, data management system 1110 can exit the synchronization stage and exchange other data synchronously with data collection apparatus 1105 over link 1130. Such a synchronization process can be repeated periodically.

In one implementation, data collection apparatus 1105 can receive and/or display a serial number or other identifier of a synchronized probe 100.

During a measurement stage, one or more probes 100 can collect data relating to the hydration of one or more monitored individuals. The probes 100 can perform data processing on the collected data, including bioelectric impedance data analysis, filtering, and, event identification.

The probes 100 can transmit data relating to the hydration monitoring (including results of processing and analyzing collected data) to one or more data collection apparatus 1105. The transmitted data can include a probe identifier that identifies the transmitting probe 100. The transmitted data can be encrypted.

Data collection apparatus 1105 can also command one or more probes 100 to transmit data relating to the hydration monitoring over link 1125. For example, data collection apparatus 1105 can transmit a query to probe 100. The query can request that probe 100 provide information regarding some aspect of the hydration monitoring. For example, a query can request that probe 100 transmit a confirmation that hydration monitoring is occurring over link 1125, a query can request that probe 100 transmit a recent measurement result over link 1125, or a query can request that probe 100 transmit one or more events of a particular character over link 1125. Data collection apparatus 1105 can transmit queries to probe 100 periodically, e.g., every hour or two.

Data collection apparatus 1105 can also relay some or all of the data transmitted from probe 100 to data management system 1110. The data can be relayed over one or more data links 1130, 1135. Data collection apparatus 1105 can relay such data directly, i.e., without performing additional analysis on the information, or data collection apparatus 1105 can perform additional processing on such before relaying a subset of the data to data management system 1110. Data collection apparatus 1105 can notify a local user that data has been relayed by displaying a data relay notice on local user output portion 1210. Alternatively, data can be relayed by data collection apparatus 1105 without notification to a local user.

Data collection apparatus 1105 can also receive user input over one or more of local user input portion 1205, wireless data communication portion 1215, and wired data communication portion 1217. The received input can identify that data collection apparatus 1105 is to transmit data to one or more probes 100 over link 1125. For example, the received input can identify that data collection apparatus 1105 is to instruct probe 100 to generate an alarm signal indicating that a monitored person suffers under a disease state. As another example, the received input can identify that data collection apparatus 1105 is to transmit a query to a probe 100 over wireless link 1125. As another example, the received input can identify that data collection apparatus 1105 is to transmit an instruction instructing probe 100 to change a parameter of the hydration monitoring, including one or more threshold values for identifying a disease state.

Data collection apparatus 1105 can also perform data processing and storage activities that supplement the data processing and storage activities of probe 100. For example, data collection apparatus 1105 can perform more extended data analysis and storage, including signal processing and analysis. For example, data collection apparatus 1105 can perform impedance analysis using model equivalent circuits to determine hydration levels at different locations in a monitored organism. As another example, data collection apparatus 1105 can perform trending analyses that identify a general tendency of hydration levels to change over extended periods of time, or data collection apparatus 1105 can perform comparisons between hydration levels obtained using multiple probes 100. The multiple probes 100 can monitor the hydration of a single organism, or the multiple probes can monitor the hydration of multiple organisms. Data collection apparatus 1105 can compare and correlate monitoring results from multiple probes to calibrate one or more probe 100 and minimize errors during monitoring.

Data collection apparatus 1105 can also compare and/or correlate the results of hydration monitoring with the results of monitoring other biological parameters. For example, data collection apparatus 1105 can compare and correlate the results of hydration monitoring with the results of heart monitoring, drug delivery schedules, and temperature monitoring. Data collection apparatus 1105 can receive the other monitoring results over one or more of local user input portion 1205, wireless data communication portion 1215, and wired data communication portion 1217. For example, data collection apparatus 1105 can receive the other monitoring results over one or more of links 1125, 1130, 1135.

Data collection apparatus 1105 can also exchange data with other devices and systems (not shown in FIG. 6). For example, data collection apparatus 1105 can receive other monitoring results directly from other monitoring instruments. As another example, data collection apparatus 1105 can transmit data relating to the results of hydration monitoring to other local or remote parties. The other parties can be external entities in that they do not share a legal interest in any of the constituent components of system 1100. For example, the other parties can be a medical group that has contracted with an owner of system 1100 to monitor hydration of an individual.

Data management system 1110 can receive the results of hydration monitoring from data collection apparatus 1105 over one or both of data link 1130, 1135. The received results can include analyses of the hydration of an organism, as well as comparisons and correlations of monitoring results from multiple organisms or other biological parameters.

Data management system 1110 can conduct operations with the received data, including processing the data to identify disease states and problems with the monitoring. For example, data management system 1110 can perform impedance analysis using model equivalent circuits to determine hydration levels at different locations in a monitored organism. As another example, data management system 1110 can perform trending analyses that identifies a general tendency of hydration levels to change over extended periods of time, or data management system 1110 can perform comparisons between hydration levels obtained using multiple probes 100. The multiple probes 100 can monitor the hydration of a single organism, or the multiple probes can monitor the hydration of multiple organisms. Data management system 1110 can compare and correlate monitoring results from multiple probes to calibrate one or more probe 100 and minimize errors during monitoring. Data management system 1110 can also perform analyses that require hydration monitoring results from statistically significant numbers of organisms. Such analyses can include billing assessments, geographic assessments, epidemiological assessments, etiological assessments, and demographic assessments.

Data management system 1110 can render the results of hydration monitoring on one or more input/output devices 1115 and store the results of hydration monitoring on one or more data storage devices 1120. Data management system 1110 can also provide the results of the data processing to data collection apparatus 1105 and/or probe 100 over data links 1125, 1130, 1135. The provided results can include an indication that a disease state is present and/or an indication that probe 100 should generate an alarm signal indicating that a monitored organism suffers under a disease state. Data management system 1110 can also provide such indications to external entities, including medical personnel interacting with input/output device 1115 and medical personnel in the vicinity of the monitored organism. As another example, data management system 1110 can also post an indication in an external system such as the clinical information system of a healthcare organization or an Internet portal.

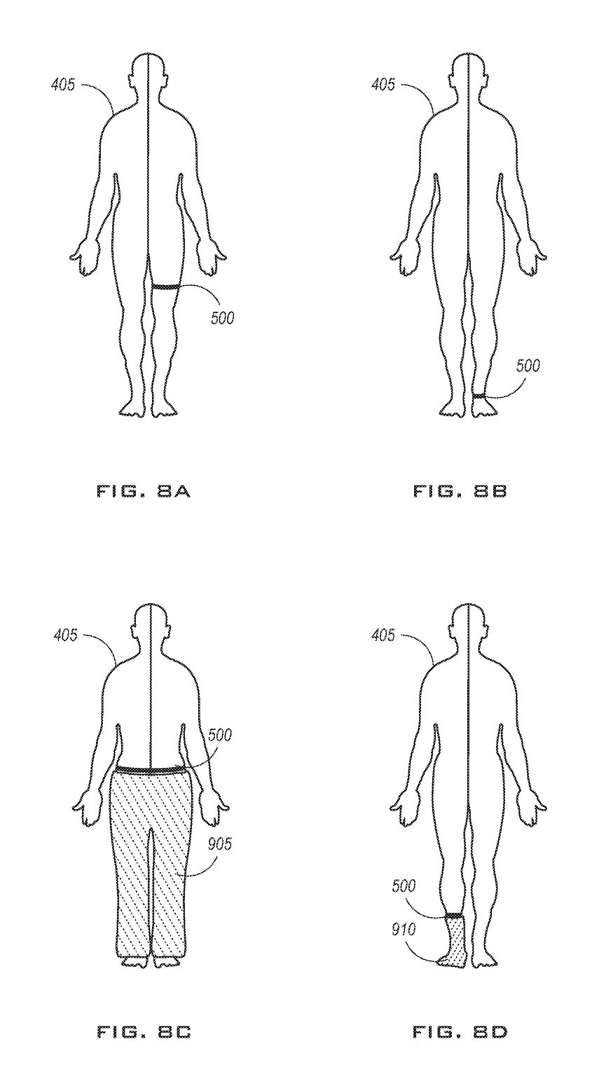

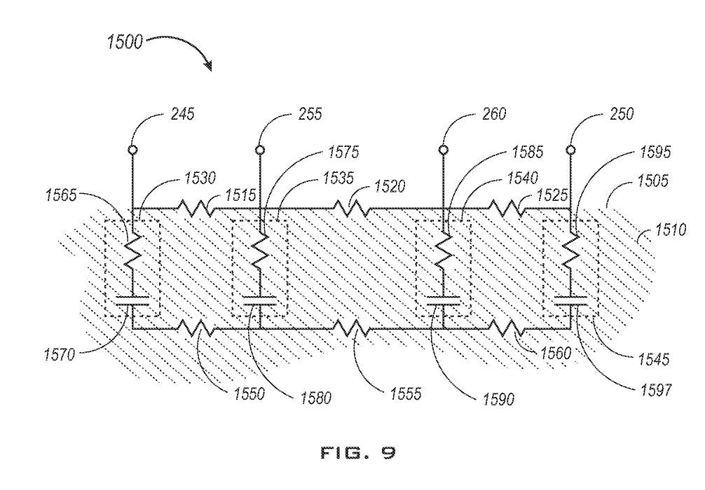

FIGS. 8A-8D illustrate example deployments of implementations of strap probe 500 to monitor hydration in a region of a diabetic person to detect susceptibility to leg and foot ulcers. In FIG. 8A, strap probe 500 is sized to encircle the thigh of person 405 and is deployed to probe the conductivity of the thigh of person 405. In FIG. 8B, strap probe 500 is sized to encircle the lower leg of person 405 and is deployed to probe the conductivity of the lower leg of person 405. As shown, strap probe 500 encircles the ankle, but strap probe 500 can also encircle the foot, the calf, or a toe to probe the conductivity of the lower leg. In FIG. 9A, strap probe 500 is incorporated into a pair of pants 905 and sized to encircle the torso of person 405 to probe the conductivity of the torso of person 405. Incorporating a probe 500 into pants 905 may reduce the intrusiveness of probe 500 and help ensure that a monitored individual deploys probe 500. In FIG. 9B, strap probe 500 is incorporated into a sock 910 and sized to encircle the lower leg of person 405 to probe the conductivity of the lower leg of person 405. Incorporating a probe 500 into sock 910 may reduce the intrusiveness of probe 500 and help ensure that a monitored individual deploys probe 500. It will be appreciated that for monitoring susceptibility and potential onset of ulcers, sores, and the like, a continuously borne probe is not necessary. Periodic impedance measurements with a hand-held probe could be performed as an alternative.

The use of hand held or otherwise not attached probe may be aided by the use of external guides, e.g. templates having fixed geometries or distances, for increasing the reproducibility of measurement location on the body. Alternatively, either naturally occurring landmarks, e.g. venous patterns, or landmarks applied to the body, e.g. temporary tattoos, may be used to help reproducibility of measurement site or probe orientation.

FIG. 10 shows an example of a model equivalent circuit 1500 that can be used to understand the effects of various tissue impedances on an overall impedance measurement. In particular, model equivalent circuit 1500 that can be used to model the electrical conductivity of an organism or local region of an organism. Circuit 1500 models the impedances using a probe 200 that supports electrodes 245, 250, 255, 260 above a skin surface 1505 of an organism 1510.

Model circuit 1500 includes a series of surface impedances 1515, 1520, 1525, a series of transdermal impedances 1530, 1535, 1540, 1545, and a series of subdermal impedances 1550, 1555, 1560. Surface impedances 1515, 1520, 1525 can model the surface electrical impedances between the relevant of electrodes 245, 250, 255, 260. Surface impedances 1515, 1520, 1525 can model both the conductivity through the surface of the skin and the conductivity through sweat and other conducting fluids on the surface of the skin. In one implementation, surface impedances 1515, 1520, 1525 are modeled as non-reactive (i.e., resistive) elements.

Transdermal impedances 1530, 1535, 1540, 1545 can model the electrical impedances through the skin of a monitored organism. Transdermal impedance 1530 includes a resistive component 1565 and a reactive component 1570. Transdermal impedance 1535 includes a resistive component 1575 and a reactive component 1580. Transdermal impedance 1540 includes a resistive component 1585 and a reactive component 1590. Transdermal impedance 1545 includes a resistive component 1595 and a reactive component 1597. Reactive components 1570, 1580, 1590, 1597 can model the electrical impedance through dense cellular layers as a capacitive element, whereas resistive components 1565, 1575, 1585, 1595 can model the electrical impedance through hydrated and other portions of the skin as a resistive element.

Subdermal impedances 1550, 1555, 1560 can model electrical impedances through a monitored organism. For example, subdermal impedances 1550, 1555, 1560 can model the electrical impedances of a portion of the monitored organism as a resistive volume conductor bounded by the skin.

In one implementation, in bioelectric impedance spectroscopy, probe 200 supports electrodes 245, 250, 255, 260 above skin surface 1505. Current source 210 can drive electrical current between electrodes 245, 250. The driven current can include both direct current and alternating current components. The potential at electrodes 245, 250, 255, 260 provides information about the net impedance across equivalent circuit 1500 as well as the impedance of different paths across equivalent circuit 1500.

For example, when direct current is driven across circuit 1500, a large portion of the direct current will pass through surface impedances 1515, 1520, 1525. Potential measurements at electrodes 245, 250, 255, 260 under direct current application can be used to estimate the impedance of surface impedances 1515, 1520, 1525. When certain frequencies of alternating current are driven through circuit 1500, some portion of the alternating current can pass through surface impedances 1515, 1520, 1525, transdermal impedances 1530, 1535, 1540, 1545, and subdermal impedances 1550, 1555, 1560. Potential measurements at electrodes 245, 250, 255, 260 can be used to estimate impedances 1515, 1520, 1525, 1530, 1535, 1540, 1545, 1550, 1555, 1560. Such estimations can be made in light of the estimations of surface impedances 1515, 1520, 1525 made using direct current.

The impact of various factors on the electrical conductivity of an organism can be accommodated by changing the mathematical analysis of model circuit 1500 or by changing aspects of data collection. For example, when surface impedances 1515, 1520, 1525 are particularly low, e.g., due to heightened conductivity through sweat or other conducting fluids on the surface of the skin, the measured potentials at electrodes 245, 250, 255, 260 can be mathematically corrected to accommodate the lowered conductivity. For example, previously obtained surface impedance estimates can be used to estimate the effect that changes in surface impedances 1515, 1520, and 1525 have on the total impedance measurement, and thus isolate the change in sub-dermal impedance so as to more accurately monitor changes in subdermal tissue hydration. Alternatively, bioelectric spectroscopy measurements can be delayed altogether or probe 200 can output an indication to a monitored individual that the individual should dry the measurement region.

Model equivalent circuit 1500 can be used in conjunction with custom approaches to data analysis for monitoring the hydration of an organism. Such data analysis approaches can be used to interpret monitoring data and to identify changes in the amount and distribution of water in a monitored organism. Data analysis approaches can also be used to incorporate results of other bioparameter measurements and responses to survey questions into the hydration monitoring. In certain implementations, such questions may be transmitted automatically to the organism from the data collection unit or the data management system. An implementation of such an automated process may include the use interactive voice response systems (IVRS) or displayed questions displayed on the data collection unit.

Data analysis approaches can be performed in accordance with the logic of a set of machine-readable instructions. The instructions can be tangibly embodied in machine-readable format on an information carrier, such as a data storage disk or other memory device. The instructions can also be embodied in whole or in part in hardware such as microelectronic circuitry.

Data analysis approaches can yield analysis results that can be displayed to a human user. The human user can be the monitored individual or another individual, such as a medical professional. The analysis results can be displayed in response to a prompt from the user or automatically, i.e., without user input. For example, the analysis results can be displayed automatically when hydration indicative of a disease state is identified. When hydration monitoring is performed using a system 1100, analysis results can be displayed at a probe 100, at a data collection apparatus 1105, and/or at a data management system 1110 (FIGS. 11, 13, 14). Analysis results can be displayed using other output devices such as the postal service, facsimile transmission, voice messages over a wired or wireless telephone network, and/or the Internet or other network-based communication modalities.

Data analysis can be performed continuously or intermittently over extended periods of time. The analyzed data can be measurement results collected continuously or intermittently. The analyzed data can be a subset of the data collected or the analyzed data can be all of the data collected. For example, the analyzed data can be intermittent samples redacted from the results of continuous hydration monitoring.

Data analysis operations can be performed at one or more of probe 100, data collection apparatus 1105, and/or data management system 1110. In one implementation, data analysis is distributed between probe 100 and data collection apparatus 1105. In particular, probe 100 can perform initial analyses, including signal processing, noise filtering, and data averaging operations. The operations can be performed on data from one or more measurements taken at one or more frequencies. The operations can be performed on raw data or on data where variations have been accommodated. For example, the operations can be performed on data collected at certain points during breathing. These initial analysis results can be transmitted, along with other information such as a probe identifier and a time/date stamp, to data collection apparatus 1105. At data collection apparatus 1105, data analysis operations can include the identification of trends or shifts in hydration associated with disease states such as pulmonary edema, as well as comparisons between received data and threshold values. In addition such analysis may include a mapping of the pre-emergent wound or wound region. Such mapping and characterization may include a graphical representation of healing pattern of the region in question and/or comparison of observed values to healthy or normally healing tissue. Such comparison may present measured data directly or representations of measured data, e.g. a healing stage index, displayed numerically or as colored domains of a graphic representation of the region in question, e.g. red=poorly healing or at risk for ulceration, green=normally healing or health tissue. Wound and/or regional status may be displayed on the probe, data collection unit and/or at the data management system.

In another implementation, data analysis operations are performed primarily at data collection apparatus 1105 and data analysis at probe 100 is minimal. When data analysis at probe 100 is minimal, data analysis and data storage can be consolidated at data collection apparatus 1105 and probe 100 can include simplified circuitry with reduced power requirements and cost.

Data analysis can also be performed at data management system 1110. Such data analysis can include multivariable analysis where hydration monitoring results are analyzed in light of other statistical variables such as weight, heart rate, respiration, time of day, month, eating patterns, physical activity levels, and other variables. The other statistical variables need not be entirely independent of the hydration monitoring results. The hydration monitoring results used in multivariable analysis can be obtained over extended periods (e.g., days, weeks, or months) from one or more organisms. The results of such multivariable analysis can be used to develop new and improved analyses of hydration monitoring results, including improved algorithms. Such analysis may also include improved pattern definition techniques, neural net analysis and/or artificial intelligence based systems.

A variety of other analysis techniques can be applied to hydration monitoring results. These include the use of established guideline values for data that is used to determine fluid changes associated with the onset or progression of pulmonary edema. Also, clinician-modified variables such as tailored threshold values can be applied to permit increased accuracy and specificity.

Data analysis can include comparisons involving recent hydration monitoring results. For example, recent hydration monitoring results can be compared with previous hydration monitoring results, predicted results, or population results. Future hydration monitoring results can be predicted based on the current state of the monitored individual and on past hydration monitoring results obtained with the same or with other individuals or a population or demographic group. Such comparisons may include, for example, the use of population data tables, multiple reference measurements taken over time, or the results of trend analyses based upon extended hydration monitoring.

Accordingly, other implementations are within the scope of the following claims.

What is claimed is:

1. A method of monitoring a wound, said method comprising measuring electrical impedance of tissue proximate to the wound at two or more times during wound healing.

2. The method of claim 1, comprising connecting two or more electrodes to skin surfaces proximate to said wound.

3. The method of claim 2, wherein the electrodes are an integral part of a wound dressing.

4. The method of claim 1, comprising comparing electrical impedance measurements obtained at different times.

5. The method of claim 1, comprising performing a therapeutic treatment or action on said wound in response to one or more impedance measurements.

6. The method of claim 1, further comprising communicating information related to measured wound status to one or both a patient with said wound and medical personnel.

7. The method of claim 1, further comprising sensing skin temperature proximate to said wound.

8. The method of claim 1, further comprising sensing heat flux from skin proximate to said wound.

9. A wound dressing comprising:
an absorbent material adapted to absorb wound exudate, and
a plurality of electrodes situated to apply an electric current and/or voltage to tissue proximate to said wound.

10. The wound dressing of claim 9, wherein said dressing additionally comprises circuitry configured to supply current through said tissue via at least two of said electrodes.

11. The wound dressing of claim 10, wherein said dressing additionally comprises circuitry configured to measure voltage across at least two of said electrodes.

12. The wound dressing of claim 9, wherein said dressing additionally comprises a wireless transmitter.

13. The wound dressing of claim 9, wherein said dressing additionally comprises a device and/or a pharmaceutical compound or agent to enhance healing or inhibit infection.

14. The wound dressing of claim 8, wherein said dressing additionally comprises one or more temperature sensors.

15. A method of reducing incidence of pressure wounds in a patient comprising detecting susceptibility to pressure wounds by measuring electrical impedance of a region of the body of said patient.

16. The method of claim 15, comprising initiating treatment in response to said measuring.

17. A method of reducing incidence of cutaneous ulcers in a patient comprising detecting susceptibility to cutaneous ulcers by measuring electrical impedance of a region of the body of said patient.

18. The method of claim 17, additionally comprising initiating treatment in response to said measuring.

19. The method of claim 17, wherein said patient is diabetic.

20. The method of claim 17, wherein said region comprises one or both lower legs of said patient.
